(19) United States
(12) Patent Application Publication
(10) Pub. No.: US 2011/0173877 A1
(43) Pub. Date: Jul. 21, 2011

(54) PROCESS FOR PREPARING A HYDROCARBON OR MIXTURE OF HYDROCARBONS

(76) Inventors: Jeroen Van Buijtenen, Amsterdam (NL); Jean-Paul Lange, Amsterdam (NL); Richard John Price, Cheshire (GB)

(21) Appl. No.: 12/972,170

(22) Filed: Dec. 17, 2010

(30) Foreign Application Priority Data
Dec. 24, 2009 (EP) 09180764.4

(51) Int. Cl. C10L 1/18; C07D 407/00

(52) U.S. Cl. 44/350; 549/472

(57) ABSTRACT

A process for preparing a hydrocarbon or mixture of hydrocarbons comprising the steps of
(i) hydrogenating furfural, 5-hydroxymethylfurfural or a mixture of furfural and 5-hydroxymethylfurfural to provide furfuryl alcohol, 2,5-furandimethanol or a mixture of furfuryl alcohol and 2,5-furandimethanol;
(ii) oligomerizing the alcohol or mixture of alcohols of step (i) in the presence of an acidic catalyst to provide a carbon-carbon coupled oligomer; and
(iii) hydrogenating the oligomer of step (ii)
   is provided. The hydrocarbons are useful as fuel blending components. Processes for controlling the oligomerization of alcohol or mixture of alcohols to optimise the production of oligomers suitable for conversion to hydrocarbons useful as kerosene and diesel components are also provided.

This application claims the benefit of European Application No. 09180764.4 filed Dec. 24, 2009 which is incorporated herein by reference.

### FIELD OF THE INVENTION

The present invention relates to a process for the preparation of a hydrocarbon or mixture of hydrocarbons. The hydrocarbon or mixture of hydrocarbons prepared by this process can be useful as components of diesel and kerosene.

### BACKGROUND TO THE INVENTION

Biofuels are combustible fuels, typically derived from biological sources, which result in a reduction of greenhouse gas emissions. Examples of biofuels used for blending with conventional gasoline fuel components include alcohols, in particular ethanol. Also biofuels such as fatty acid methyl esters derived from rapeseed and palm oil can be blended with conventional diesel components for use in diesel engines. However, these are generally more expensive to produce than ethanol.

Alternatives to ethanol which have been investigated as biofuels include furan-based components, such as methylfuran, as described in US 20090234142, which can potentially be produced from cellulose material with less complex processing than would be needed for ethanol. A disadvantage associated with the use of such furan based components, however, is the fact that they contain oxygen and, therefore, deliver less energy per liter of fuel than do hydrocarbons upon combustion. Moreover, their low boiling point does not allow blending in kerosene and diesel.

There therefore remains a continuing need for the development of alternative components which can be derived from biological sources and blended with conventional kerosene and diesel fuel components for use in fuel formulations.

### SUMMARY OF THE INVENTION

In an embodiment, a process for preparing a hydrocarbon or mixture of hydrocarbons is provided comprising the steps of
(i) hydrogenating furfural, 5-hydroxymethylfurfural or a mixture of furfural and 5-hydroxymethylfurfural to provide furfuryl alcohol, 2,5-furandimethanol or a mixture of furfuryl alcohol and 2,5-furandimethanol;
(ii) oligomerizing the alcohol or mixture of alcohols of step (i) in the presence of an acidic catalyst to provide a carbon-carbon coupled oligomer; and
(iii) hydrogenating the oligomer of step (ii).

Hydrocarbons can be prepared starting from materials which are readily obtainable from biomass, which hydrocarbons can advantageously be used as fuel components.

In a further embodiment, a process for preparing a C₉-C₂₀ hydrocarbon is provided comprising hydrogenating an oligomer of furfuryl alcohol, 2,5-furandimethanol or a mixture of furfuryl alcohol and 2,5-furandimethanol.

### BRIEF DESCRIPTION OF THE DRAWINGS

FIG. 1 shows a schematic diagram of a process according to the invention.

### DETAILED DESCRIPTION OF THE INVENTION

In step (i) of the process of the first aspect of the invention furfural, 5-hydroxymethylfurfural or a mixture of furfural and 5-hydroxymethylfurfural is hydrogenated to provide furfuryl alcohol, 2,5-furandimethanol or a mixture of furfuryl alcohol and 2,5-furandimethanol.

Where the starting material is furfural, this furfural may be obtained from any suitable source. It may, for example, suitably be obtained from lignocellulosic biomass by acid-catalysed hydrolysis and/or dehydration of pentosan rich feedstocks such as corncobs, rice husk and sugarcane bagasse.

Where the starting material is 5-hydroxymethylfurfural, this may conveniently be obtained in similar manner from hexose/hexosan rich feedstocks such as glucose, starch or lignocellulose.

Hydrogenation of furfural to give furfuryl alcohol may be carried out under conventional gas or liquid-phase hydrogenation conditions using a suitable catalyst such as CuCr as described for example in chapter 17, pages 150-155 of the handbook by K. J. Zeitsch, titled “The chemistry and technology of furfural and its many byproducts”, published by Elsevier Science B.V., 2000.

Hydrogenation of 5-hydroxymethylfurfural or its mixture with furfural may similarly be carried out under conventional liquid-phase hydrogenation conditions to give 2,5-furandimethanol (see Chapter 7 of G. C. A. Luijkx, PhD Thesis at the Technical University of Delft, the Netherlands, 1994).

In a preferred embodiment furanic components such as furfural and 5-hydroxymethylfurfural are obtainable from lignocellulosic biomass by acid-catalysed dehydration of pentoses and hexoses respectively.

In step (ii) of the process of the first aspect of the invention the alcohol or mixture of alcohols of step (i) is oligomerized in the presence of an acidic catalyst to provide a carbon-carbon coupled oligomer.

By a carbon-carbon coupled oligomer is herein understood an oligomer comprising two or more monomers, which monomers are coupled to each other via a carbon-carbon bond.

Polymerization of furfuryl alcohol in the presence of an acidic catalyst such as a dilute aqueous acidic catalyst is described in British patent No. 682,666. The composition of the polymeric product so produced is not reported in this British patent. The formation of low molecular weight oligomers under acid-catalysed conditions has been reported for example by Wewerka et al., J. Applied Polymer Science, 1971, Vol. 15, pages 1437-1451 and by Barr et al, J. Applied Polymer Science, 1971, vol. 15, pages 1079-1090.

The oligomerization steps according to the first and second aspects of the invention may conveniently be effected using any acidic catalyst material, which is suitably resistant to the process conditions used and in any suitable solvent.

In one embodiment the acidic catalyst is dissolved in the reaction mixture, which reaction mixture can for example contain furfural, 5-hydroxymethylfurfural, furfuryl alcohol, 2,5-furandimethanol and/or water. The dissolved acidic catalyst is preferably present in an amount of equal to or more than 0.0001 wt %, more preferably equal to or more than 0.001 wt %, and most preferably equal to or more than 0.01 wt %, and preferably equal to or less than 10 wt %, more preferably equal to or less than 1 wt % of acid, and most preferably equal to or less than 0.1 wt % based on the total weight of reaction mixture present.

In one embodiment, the acidic catalyst comprises an aqueous acidic catalyst. By an aqueous acidic catalyst is herein understood a catalyst which can be dissolved in water (is water-soluble). Suitable water-soluble acidic materials which can be used include aqueous mineral acids, for example aqueous phosphoric, hydrochloric, sulphuric or p-toluene sulphonic acid or mixtures thereof, or organic acids such as formic and acetic acid. In one particular embodiment, the aqueous acidic catalyst comprises aqueous sulphuric acid.

Suitably where the acidic catalyst comprises an aqueous acidic catalyst, this is a dilute aqueous acidic catalyst. In a preferred embodiment the acidic catalyst concentration is therefore in the range of from 0.0001 wt % to 0.1 wt %, based on the total weight of reaction mixture present.

We have found that the use of a dilute aqueous acidic catalyst combined with the formation of a separate product phase (in which the acid is poorly soluble) during reaction as a result of poor solubility of the hydrophobic oligomers in water is advantageous as it restricts the rate of the oligomerization reaction and hence affords the possibility of controlling the oligomerization to limit the conversion of the alcohol. In this way, the formation of insoluble resins can be minimized.

In a preferred embodiment step (ii) therefore comprises oligomerizing the alcohol or mixture of alcohols of step (i) in the presence of an aqueous acidic catalyst to provide a first hydrophobic phase comprising carbon-carbon coupled oligomer and a second aqueous phase comprising aqueous acidic catalyst. The two phases can advantageously be separated via phase separation. If desired, the separated aqueous acidic catalyst can be recycled.

Typically where a strong acidic catalyst such as H₂SO₄ is employed in dilute aqueous solution, the catalyst concentration is in the range of 0.0001 to 0.1 wt %, preferably 0.001 to 0.01 wt %. It will be appreciated that higher catalyst concentrations should be used where weaker acids are used as catalysts.

Oligomers of desired chain length can be recovered from unconverted furfuryl alcohols and heavy oligomers by conventional separation technologies such as atmospheric or vacuum distillation or membrane separation.

In one embodiment of the process according to the first aspect of the invention, the hydrogenation step (i) and oligomerization step (ii) may conveniently be integrated by utilizing an acid-resistant hydrogenation catalyst and feeding the hydrogenation reactor with hydrogen, furfural and an amount of acid, for example sulphuric acid. Preferred are amounts of acid as mentioned above.

Alternatively, the hydrogenation step (iii) and oligomerization step (ii) may conveniently be integrated by placing a solid hydrogenation catalyst in, or feeding an homogeneous hydrogenation catalyst to, the oligomerization reactor. Integration of these steps affords the possibility of controlling the selectivity as the oligomers are rendered unreactive after ring hydrogenation (i.e.

hydrogenation of the furanic ring(s) of the carbon-carbon coupled oligomers). The present inventors have found that controlling the alcohol oligomerization so as to limit the alcohol conversion to no more than 95% advantageously optimises the yield of carbon-carbon coupled oligomers comprising from 2 to 4 furan rings. For practical purposes such alcohol conversion can conveniently be determined by determining the mol % of one or more specific alcohol(s) converted based on the total amount such alcohol(s) in the feed.

In one embodiment, the oligomerization of furfuryl alcohol can suitably be controlled to produce carbon-carbon coupled oligomers of formula (I) and/or (II)

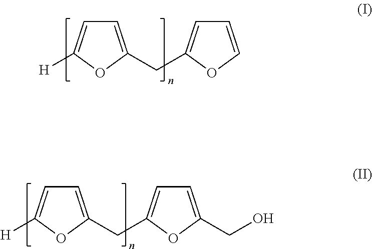

wherein n is an integer from 1 to 3, suitably 1, 2 or 3, and most preferably 2 or 3.

It will be appreciated that oligomers having different structural detail may be produced where 2,5-furandimethanol is included in the oligomerization mixture. In an embodiment where 2,5-furandimethanol is included in the oligomerization mixture, also carbon-carbon coupled oligomers of formula (III) are included:

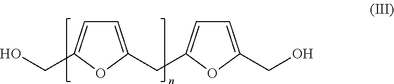

wherein n is an integer from 1 to 3, suitably 1, 2 or 3, and most preferably 2 or 3.

In this way, the formation of heavier molecular weight oligomeric products, including products having n of more than 3, can be minimized.

Any heavy products formed in the process according to the present invention can be converted to diesel, kerosene and gasoline fraction by conventional refining technologies such as catalytic cracking or hydrocracking.

Preferably the alcohol conversion in the process according to the invention is in the range of from 20 to 95%, more preferably 30 to 80%. For practical purposes such alcohol conversion can conveniently be determined by determining the mol % of one or more specific alcohol(s) converted based on the total amount such alcohol(s) in the feed.

According to another aspect, therefore, the invention provides a process for oligomerizing furfuryl alcohol, 2,5-furandimethanol or a mixture of furfuryl alcohol and 2,5-furandimethanol to prepare one or more carbon-carbon coupled oligomers comprising from 2 to 4 furan rings, which process comprises contacting the alcohol or mixture of alcohols with an acidic catalyst wherein the alcohol conversion is in the range of from 20 to 95%, preferably 30-80%.

Suitably, the reaction is carried out such as to allow the oligomers, which have a higher density than water, to separate from the aqueous phase and to trickle down the reactor. The oligomers are then preferably recovered at the bottom of the reactor while the aqueous phase and unconverted alcohol is recovered from the top of the reactor. Alternatively, the reactor can be operated in down-flow mode with both oligomers and aqueous phase being recovered from the bottom of the reactor. The oligomers can then be separated from the aqueous phase and unconverted alcohol or alcohols by simple decantation.

In one embodiment the selectivity of the oligomerization step may be shifted towards shorter chain oligomers by extracting the oligomers from the aqueous phase during the oligomerization step, by co-feeding a suitable extractant. Hence, in a preferred embodiment the oligomer product of step (ii) is extracted from the aqueous phase using an extraction solvent, preferably methyltetrahydrofuran, anisole or toluene.

In one embodiment, the extraction solvent comprises recycled oligomers, suitably after stabilization by partial hydrogenation.

In another embodiment, 5-hydroxymethylfurfural may be fed to the alcohol or mixture of alcohols in the oligomerization step.

In another embodiment, the acidic catalyst may comprise a heterogenous solid acidic material. Examples of such heterogenous solid acidic materials include ion-exchange resins; zeolites; acidic metal oxides, such as alumina or silica-alumina; or solid materials onto which acid groups have been anchored or deposited. Sulphonated styrene-divinyl-benzene resins are particularly suitable for this application.

The present inventors have advantageously found that by controlling the oligomerization of the alcohol or mixture of alcohols in step (ii) to limit the conversion of the alcohol or alcohols so as to optimise the production of carbon-carbon coupled oligomers comprising from 2 to 4 furan rings, C₉-C₂₀ hydrocarbons suitable for use as kerosene and diesel components can be produced without the need for further catalytic cracking or hydrocracking steps.

As indicated above, in one embodiment step (ii) can provide a product mixture containing unreacted furfuryl alcohol and/or unreacted 2,5-furandimethanol; C9-C20 carbon-carbon coupled oligomers; and C20+ carbon-carbon coupled oligomers. Preferably the C9-C20 carbon-carbon coupled oligomers are separated from the remainder of the product mixture and hydrogenated in step (iii). Unreacted furfuryl alcohol and/or unreacted 2,5-furandimethanol are preferably separated and recycled to step (ii) for further oligomerization. The remaining C20+ carbon-carbon coupled oligomers can advantageously be fed into a catalytic cracking or hydrocracking unit.

The separation of one or more of unreacted furfuryl alcohol and/or unreacted 2,5-furandimethanol; C9-C20 carbon-carbon coupled oligomers; and/or C20+carbon-carbon coupled oligomers from any product mixture can be carried out by various methods. For example fractions of unreacted furfuryl alcohol and/or unreacted 2,5-furandimethanol; C9-C20 carbon-carbon coupled oligomers; and/or C20+ carbon-carbon coupled oligomers may be separated by distillation in one or more distillation columns.

In a preferred embodiment the separation of one or more of unreacted furfuryl alcohol and/or unreacted 2,5-furandimethanol; C9-C20 carbon-carbon coupled oligomers; and/or C20+ carbon-carbon coupled oligomers from any product mixture is carried out with the help of one or more membranes. For example, unreacted furfuryl alcohol and/or unreacted 2,5-furandimethanol and/or optionally water can be separated from the carbon-carbon coupled oligomers by a ceramic membrane(for example a TiO₂ membrane) or a polymeric membrane(for example a Koch MPF 34 (flatsheet) or a Koch MPS-34 (spiral wound) membrane). The C9-C20 carbon-carbon coupled oligomers and the C20+ carbon-carbon coupled oligomers can conveniently be separated from each other with for example a polymer grafted ZrO₂ membrane.

The use of membranes for these separations can advantageously improve the energy efficiency of the process.

In step (iii) the oligomer(s) of step (ii) are hydrogenated to suitably prepare a hydrocarbon or mixture of hydrocarbons.

Oligomers of furfuryl alcohol, 2,5-furandimethanol or a combination of the two can be hydrogenated to give hydrocarbons suitable for use as diesel and kerosene components.

Hydrogenation of the carbon-carbon coupled oligomers to give hydrocarbons may be effected using conventional hydrogenation conditions using a suitable catalyst such as group 8-11 metals supported on suitable material, such as SiO₂, Al₂O₃, TiO₂, ZrO₂, MgO, Nb₂O₅ and mixture thereof, including amorphous silica-alumina, zeolites and clays, or carbon.

The process according to the invention allows one to prepare a hydrocarbon or a mixture of hydrocarbons. In one preferred embodiment such hydrocarbon or mixture of hydrocarbons essentially consist(s) of hydrocarbons consisting of carbon, hydrogen and optionally oxygen. In another preferred embodiment such hydrocarbon or mixture of hydrocarbons essentially consist(s) of hydrocarbons consisting of carbon and hydrogen. Most preferably the hydrocarbon or mixture of hydrocarbons consists for at least 95 wt %, more preferably at least 99 wt % of hydrocarbons consisting of carbon and hydrogen.

Depending on the hydrogenation conditions employed, the oligomers, especially the oligomers derived from furfuryl alcohol, can either undergo ring opening or hydrogenation can be halted at an intermediate stage to provide intermediates in which the furan rings are saturated to form tetrahydrofuran rings.

In one embodiment at least part of the carbon-carbon coupled oligomers in step (iii) undergo ring opening and are converted to provide one or more alkanes. Such one or more alkanes may include straight, branched and cyclo alkanes. In a preferred embodiment step (iii) provides a composition consisting essentially of C9-C20 alkanes. More preferably such a composition contains at least 20 wt % of branched alkanes.

In another embodiment at least part of the carbon-carbon coupled oligomers in step (iii) merely undergo saturation of the furanic rings to form hydrocarbon(s) consisting essentially of carbon, hydrogen and oxygen, such as for example oligomers containing one or more tetrahydrofuran rings.

In still another embodiment at least part of the carbon-carbon coupled oligomers in step (iii) may in addition undergo ring-rearrangement to form oligomers containing one or more tetrahydropyran rings.

In one further embodiment one or more of the above may be combined such that in step (iii) a mixture of C9-C20 alkanes and/or oligomers containing tetrahydrofuran rings and/or oligomers containing tetrahydropyran rings may be produced.

For instance, I. F. Bel'skii and N. I. Shuikin (Russian Chemical Reviews (English edition), 1963, 32(6), 307-321) report that furfural, furfuryl alcohol and alkyl furans undergo mainly ring-hydrogenation over supported Ni catalysts at 100° C. or over supported Pd catalysts at 275° C. In contrast, the same components undergo mainly ring-cleavage over supported Pt and Ru catalysts at 200-300° C.

The prepared hydrocarbon or mixture of hydrocarbons can be suitable for use as components of diesel and kerosene.

In one embodiment, saturated ring products (also referred to herein as saturated intermediates) of formula (IV) may be formed

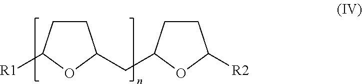

wherein n is an integer from 1 to 3, suitably 1, 2 or 3, and most preferably 2 or 3; wherein R1 is H, —CH₂OH or —CH₃; and wherein R2 is H, —CH₂OH or —CH₃.

In one preferred embodiment at least one of R1 and R2 is —CH₃ to allow for higher energy density of the product. In another embodiment at least one of R1 and R2 is —CH₂OH which can be suitably used for subsequent esterification and/or etherification as described herein below.

In a preferred embodiment, saturated ring products (also referred to herein as saturated intermediates) of formula (V)

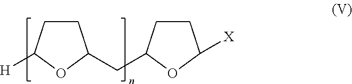

wherein n is an integer from 1 to 3, suitably 1, 2 or 3, and most preferably 2 or 3 and X is H, —CH₂OH or —CH₃, may suitably be formed.

In a further embodiment, saturated ring products (also referred to herein as saturated intermediates) of formula (VI)

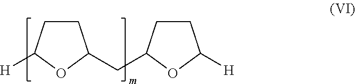

wherein m is 2 or 3, may be formed

The saturated ring products of formula (IV), (V) and (VI) are themselves of interest as diesel components and form a further aspect of the invention.

In one preferred embodiment, carbon-carbon coupled oligomers of formula (II) or (III) that can be prepared in oligomerization step (ii) are forwarded to an intermediate etherification step or an intermediate esterification step before being hydrogenated in step (iii).

In another preferred embodiment saturated ring products of formula (IV) or (V) that comprise at least one —CH₂OH group are forwarded to a subsequent etherification step or a subsequent esterification step. Of these, etherification is preferred as an intermediate, respectively subsequent step. If desired, in a preferred embodiment, etherification can be carried out in-situ during step (ii) by having an alkanol present during the oligomerization. The alkanol is preferably a C1-C6 alkanol, more preferably a C1-C4 alkanol, and still more preferably methanol, ethanol, n-propanol, isopropanol, n-butanol, tert-butanol, or isobutanol. Most preferably the alkanol is ethanol or methanol.

Hence, in one preferred embodiment step (ii) comprises oligomerizing the alcohol or mixture of alcohols of step (i) in the presence of an acidic catalyst to provide a carbon-carbon coupled oligomer of formula (II) or (III) and reacting the carbon-carbon coupled oligomer of formula (II) or (III) to convert one or more —CH₂OH group(s) into one or more ether and/or ester group(s). In such an embodiment advantageously carbon-carbon coupled oligomers of formula (VII) can be formed,

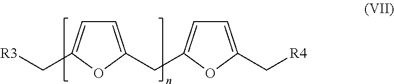

wherein n is an integer from 1 to 3, suitably 1, 2 or 3, and most preferably 2 or 3; wherein R3 is hydrogen, an —O—R group or an —O—C(O)—R group, wherein R is an alkyl group; and wherein R4 is an —O—R group or an —O—C(O)—R group, wherein R is an alkyl group.

R3 and R4 may be the same or different. Preferably the alkyl group R represents a C1-C6 alkyl, more preferably a C1-C4 alkyl and still more preferably methyl, ethyl, n-propyl, isopropyl, n-butyl, tert-butl, or isobutyl. R3 and R4 may contain the same or a different alkyl group.

In a preferred embodiment the —O—R group (ether) is a methyl-ether(methoxy-), ethyl-ether(ethoxy-), propyl-ether(propoxy-), butyl-ether(butoxy-), pentyl-ether(pentoxy-) or hexyl-ether(hexoxy-).

In another preferred embodiment the —O—C(O)—R group (ester) is a methanoate (—O—C(O)-Me), ethanoate (—O—C(O)-Et), propanoate (—O—C(O)—Pr), butanoate (—O—C(O)-Bu), pentanoate (—O—C(O)-Pe) or hexanoate (—O—C(O)-Hx).

In another preferred embodiment step (iii) comprises hydrogenating the oligomer of step (ii) to provide a product of formula (IV) that comprises one or more —CH₂OH group(s); and reacting the product of formula (IV) that comprises one or more —CH₂OH group(s) to convert such one or more —CH₂OH group(s) into one or more ether and/or ester group(s). In such an embodiment advantageously saturated ring products of formula (VIII) can be formed,

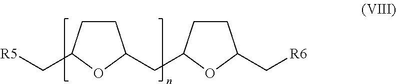

wherein n is an integer from 1 to 3, suitably 1, 2 or 3, and most preferably 2 or 3; wherein R5 is hydrogen, an —O—R group or an —O—C(O)—R group, wherein R is an alkyl group; and wherein R6 is hydrogen, an —O—R group or an —O—C(O)—R group, wherein R is an alkyl group; with the proviso that at least one of R5 and R6 is an —O—R group (ether) or an —O—C(O)—R (ester) group, wherein R is an alkyl group. R5 and R6 may be the same or different. Preferably the alkyl group R represents a C1-C6 alkyl, more preferably a C1-C4 alkyl and still more preferably methyl, ethyl, n-propyl, isopropyl, n-butyl, tert-butl,or isobutyl as defined herein before. R5 and R6 may contain the same or a different alkyl group.

In a preferred embodiment the —O—R group (ether) is a methyl-ether(methoxy-), ethyl-ether(ethoxy-), propyl-ether(propoxy-), butyl-ether(butoxy-), pentyl-ether(pentoxy-) or hexyl-ether(hexoxy-) as defined herein before.

In another preferred embodiment the —O—C(O)—R group (ester) is a methanoate (—O—C(O)-Me), ethanoate (—O—C(O)-Et), propanoate (—O—C(O)—Pr), butanoate (—O—C(O)-Bu), pentanoate (—O—C(O)-Pe)or hexanoate (—O—C(O)-Hx) as defined herein before.

Conversion of the one or more —CH₂OH group(s) into one or more ether and/or ester group(s) can be achieved by any method known to the skilled person to be suitable for this purpose.

One preferred method comprises reaction of the one or more —CH₂OH group(s) with an alkene to form an ether.

Another preferred method comprises transesterification of the one or more —CH₂OH group(s) with an ester or a derivative thereof to provide another ester.

A still further preferred method comprises reaction of the one or more —CH₂OH group(s) with a hydroxyl-group containing compound to prepare an ether or an ester. By a hydroxyl-group containing compound is herein understood a compound comprising an -OH group. Preferably the one or more hydroxyl-group containing compound(s) comprise alkanol(s) and/or carboxylic acid(s). More preferably the hydroxyl-group containing compound is an alkanol, preferably a C1-C6 alkanol, more preferably a C1-C4 alkanol, and still more preferably methanol, ethanol, n-propanol, isopropanol, n-butanol, tert-butanol, or isobutanol. Most preferably the hydroxyl-group containing compound is ethanol or methanol.

Preferably conversion of the one or more —CH₂OH group(s) into one or more ether group(s) (that is etherification) and/or into one or more ester group(s) (that is esterification) is carried out in the presence of an acidic or basic catalyst. Catalysts that can be used for the etherification and/or esterification include for example H₂SO₄, HCl, W/ZrO2, para-toluenesulfonic acid (pTSA), as well as acidic ion-exchange resins such as for example Amberlyst 15, sulphonated oxides and zeolites.

Etherification can be carried out at any temperature and pressure known by the skilled person to be suitable for an etherification reaction. Preferably, however, etherification is carried out at a temperature equal to or more than 50° C., preferably equal to or more than 100° C. and equal to or less than 300° C., more preferably equal to or less than 250° C., most preferably equal to or less than 200° C.

Also esterification can be carried out at any temperature and pressure known by the skilled person to be suitable for an esterification reaction. Preferably, however, esterification is carried out at a temperature equal to or more than 50° C. and equal to or less than 250° C., more preferably equal to or less than 150° C., most preferably equal to or less than 100° C.

The ethers and esters provided by the above process are believed to have improved density characteristics, allowing for improved blending of such ethers and esters with base fuels. The saturated ring products of formula (VIII) are therefore of interest as fuel components, and in specific as diesel components, and form a further aspect of the invention.

Without wishing to be bound by any kind of theory, it is believed that the esterification and/or etherification as described above may lead to an improved cetane number, reduced density, increased energy density and/or reduced polarity.

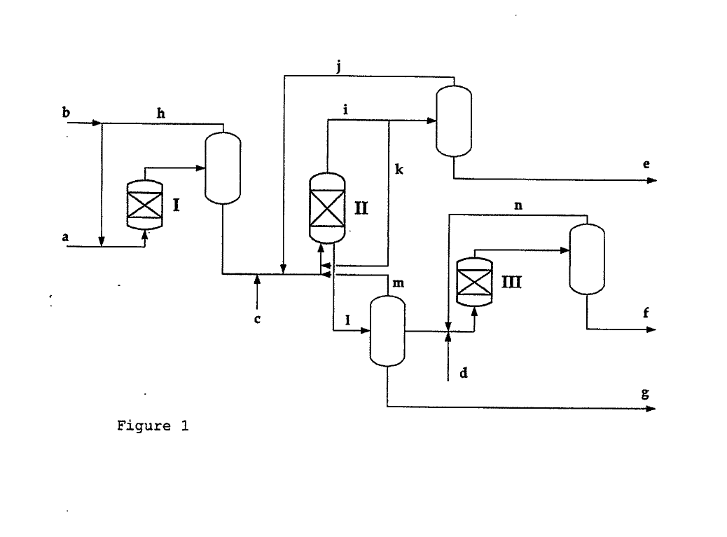

FIG. 1 shows a process scheme of a process according to the invention. In FIG. 1 furfural is introduced into a system at (a), for example at around 92% by weight in water. Hydrogen is introduced at (b) and these two reactants pass into a furfural hydrogenation reactor (I) which contains a hydrogenation catalyst.

The output from the reactor (I) is separated (for example in a gas-liquid separator (S1)) into unreacted hydrogen which is recycled via (h) and a liquid phase output, including furfuryl alcohol hydrogenation product, which is passed to a oligomerization reactor (II). An aqueous acid oligomerization catalyst, such as dilute sulphuric acid can be introduced at (c), and passed to the oligomerization reactor (II).

In the oligomerization reactor (II) the C₉-C₂₀ oligomer can form a separate product phase, which will also contain some unreacted furfuryl alcohol as well as heavier oligomer by-products. This product phase—being denser than the aqueous phase, can conveniently be removed from the bottom of the oligomerization reactor (II) and conveyed by line (1), away from the oligomerization reactor (II). The product phase from line (1) is forwarded to a separator (S2) where unreacted furfural is separated out and returned via line (m) to the oligomerization reactor (II), while the heavy products are removed via (g).

The aqueous phase, which includes furfuryl alcohol, water and sulphuric acid, is removed at the top of the oligomerization reactor (II), via line (i), and at least partly recycled to the reactor to allow further opportunity for reaction, via line (k). Another part is not recycled but instead forwarded to a distillation column (D1). Via the distillation column (D1) part of the water and furfuryl is returned to the oligomerization reactor as an azeotropic mixture (j), which is distilled from purge (e), consisting of water produced in the oligomerization and water introduced with the feedstock together with some dissolved acid.

The C₉-C₂₀ oligomer is forwarded from the separator (S2), together with hydrogen introduced at (d), to the hydrogenation reactor (III). In this reactor C₉-C₂₀ hydrocarbons are formed. The product of the hydrogenation reactor (III) is forwarded to another separator (S3). In this separator (S3) C₉-C₂₀ hydrocarbons are removed via line (f). Unreacted hydrogen is returned to the hydrogenation reactor (III) via line (n), to provide further opportunity for it to react.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples.

Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith.

Moreover unless stated otherwise, any feature disclosed herein may be replaced by an alternative feature serving the same or a similar purpose.

### EXAMPLES

The invention will now be further illustrated by means of the following non-limiting examples.

An aqueous solution of furfuryl alcohol was used as a simulation for the product of a hydrogenation of furfural.

Experiments to oligomerise furfuryl alcohol were performed under batch or continuous conditions according to the following general methods:

### (i) Batch Experiment

Distilled furfuryl alcohol, water and optionally an extractant were added to a round bottom flask. Stirring was started and the flask was subsequently heated to the desired reaction temperature. The reaction was started by adding a solution of H₂SO₄ in water. Samples for GC analysis were taken during the reaction and the reaction was terminated after the desired evidence time by cooling with water and dissolving the heterogeneous product in a known amount of ethanol and dichloromethane to enable analysis of a homogeneous combined product phase.

### (ii) Continuous Experiment

A furfuryl alcohol solution in water, containing H₂SO₄ was pumped through a glass column (upflow or downflow), packed with glass beads. The glass column was heated to the desired reaction temperature by pumping heating oil through the mantle. A separate oil phase was formed with a higher density than water.

In the downflow experiment, feedstock enters from the top of the reactor, heavier product phase is formed and both organic and aqueous phase leave the reactor from the bottom. The combined product is neutralized with Na₂CO₃ and the organic and aqueous phases are separated.

Specific conditions used and the results obtained are given in Table 1. Unless otherwise noted, experiments were performed with aqueous solutions of 35wt % furfuryl alcohol, in batch mode and with no extractant. Conversion of furfuryl alcohol and yield of oligomers was determined by GC-FID. The oligomer yield includes both oligomers with structures (I) and (II). The yield of heavy byproducts, which are not detected with GC-FID but are identified with Size Exclusion Chromatography, was quantified based on mol balance.

The dimer obtained as product in the process according to the invention can be used as kerosene fuel component, the trimer obtained as product in the process according to the invention can be used as diesel fuel component. The molar ratio of dimer to trimer (dimer/trimer mol ratio) is representative for the molar ratio of a kerosene fuel component to diesel fuel component obtained for the product.

### Example 1

Oligomerization of furfuryl alcohol was performed with an aqueous substrate solution containing 35wt % furfuryl alcohol and in the range from 0.01wt % to 0.001 wt % H₂SO₄. Oligomerization was performed at temperatures between 75 and 100° C. and residence times between 0.7 and 24 hours Conditions are summarized in table 1, Example No. 1.1-1.8. Furfuryl alcohol conversions ranged between 38 and 99 mol %, based on moles furfuryl alcohol, and the selectivity to C₉-C₂₀ oligomers was generally between 50 and 60 mol % for conversions up to 93 mol % and declined rapidly at conversions around 95 mol % or higher. The ratio between dimeric and trimeric oligomers declines with increasing conversion, indicating that the average chain length of C₉-C₂₀ oligomers increases with furfuryl alcohol conversion as expected. Results obtained from continuous experiments operated in downflow mode (Example No. 1.2-1.3) are in line with results from batch experiments.

### Example 2

Oligomerization of furfuryl alcohol was performed with an aqueous substrate solution containing between 12 wt % and 100 wt % furfuryl alcohol (FAlc.) and 0.001 wt % H₂SO₄. Specific conditions are indicated in table 1(Example No. 2.1-2.3). It was found that with a substrate concentration of 12 wt % furfuryl alcohol, selectivity to C₉-C₂₀ oligomers was only 28 mol %, based on moles furfuryl alcohol, at 83 mol % conversion (based on total moles furfuryl alcohol converted). Selectivity to C₉-C₂₀ oligomers at substrate concentrations of 70 and 100 wt % is broadly in line with results at a concentration of 35 wt %.

An example with an aqueous substrate solution containing 35 wt % furfuryl alcohol (FAlc.) and 0.11 wt % acetic acid as catalyst (Example No. 2.4) shows that similar results are obtained with an organic acid as with H₂SO₄. The pH of the aqueous phase of this experiment corresponds to a H₂SO₄ concentration of 0.001 wt %.

### Example 3

Oligomerization of furfuryl alcohol was performed with an aqueous substrate solution containing 35 wt % furfuryl alcohol and between 0.001 to 0.9 wt % H₂SO₄ in presence of an organic solvent as extractant. The organic solvent, used as extractant was present in an amount such that the mass (wt %) of extractant is equal to the mass of water (Example No. 3.1-3.5). Selectivity to C₉-C₂₀ oligomers is similar to selectivities obtained at similar conversion without extraction. In contrast, the ratio between dimeric and trimeric oligomers is significantly higher than in an experiment at similar conversion without extraction. Example No. 3.4 and 3.5 again show a dramatic decline of the selectivity to C₉-C₂₀ oligomers at conversion >95%.

### Example 4

Oligomerization of furfuryl alcohol was performed with an aqueous substrate solution containing 35wt % furfuryl alcohol. In example 4.1, 0.002 wt % (based on the total reaction mixture)of AMBERLYST 15 (A15 commercially obtainable from Rohm and Haas, AMBERLYST is a trademark), an ion-exchange resin, was submerged in the aqueous substrate solution. In example 4.2, 0.36 wt % (based on the total reaction mixture) of amorphous silica alumina (ASA X-600 commercially obtained from Criterion) was submerged in the aqueous substrate solution.

Oligomerization was performed at a temperature of 75° C. and a residence time of 24 hours. Conditions are summarized in table 1, Examples No. 4.1 and 4.2.

### Example 5

Oligomerization of furfuryl alcohol was performed at an aqueous substrate solution containing 35wt % furfuryl alcohol and 0.013 wt % of Re₂O₇. Oligomerization was performed at a temperature of 75° C. and a residence time of 24 hours. Conditions are summarized in table 1, Example No. 5.1.

TABLE 1
Conditions and results for examples 1 to 5

| | | | | | | Yield | Heavy | Select. | dimer/ | |
|---|---|---|---|---|---|---|---|---|---|---|
| Example | Residence | Temp | | Acid | Conv. | oligomers | products | oligomers | trimer | |
| No. | time [h] | [° C.] | Acid | [wt %] | [mol %] | [mol %] | [mol %] | [mol %] | ratio | |
| 1.1 | 7.0 | 75 | H₂SO₄ | 0.001 | 38 | 22 | 14 | 58 | 2.2 | batch |
| 1.2 | 0.7 | 90 | H₂SO₄ | 0.01 | 45 | 28 | 17 | 61 | 1.1 | continuous, downflow |
| 1.3 | 2.1 | 90 | H₂SO₄ | 0.01 | 76 | 41 | 34 | 54 | 0.8 | continuous, downflow |
| 1.4 | 4.5 | 75 | H₂SO₄ | 0.01 | 79 | 37 | 40 | 47 | 1.4 | batch |
| 1.5 | 2.8 | 100 | H₂SO₄ | 0.001 | 88 | 50 | 31 | 56 | 0.7 | batch |
| 1.6 | 24 | 75 | H₂SO₄ | 0.001 | 93 | 50 | 37 | 54 | 0.7 | batch |
| 1.7 | 8.8 | 75 | H₂SO₄ | 0.01 | 94 | 40 | 51 | 42 | 0.7 | batch |
| 1.8 | 5.9 | 100 | H₂SO₄ | 0.001 | 99 | 38 | 55 | 38 | 0.4 | batch |
| 2.1 | 8.8 | 75 | H₂SO₄ | 0.001 | 83 | 23 | 58 | 28 | 0.5 | batch, 12 wt % FAIc |
| 2.2 | 9.9 | 75 | H₂SO₄ | 0.001 | 56 | 24 | 29 | 44 | 1.8 | batch, 70 wt % FAIc |
| 2.3 | 5.2 | 75 | H₂SO₄ | 0.001 | 33 | 17 | 11 | 53 | 1.8 | batch, 100 wt % FAIc |
| 2.4 | 24 | 75 | acetic acid | 0.11 | 81 | 49 | 28 | 60 | 1.1 | batch, 35 wt % FAlc |
| 3.1 | 48 | 75 | H₂SO₄ | 0.01 | 78 | 46 | 21 | 60 | 2.5 | batch, extractant = MTHF |
| 3.2 | 48 | 75 | H₂SO₄ | 0.001 | 95 | 51 | 35 | 54 | 1.5 | batch, extractant = anisole |
| 3.3 | 24 | 75 | H₂SO₄ | 0.001 | 95 | 52 | 37 | 54 | 1.2 | batch, extractant = toluene |
| 3.4 | 2.3 | 75 | H₂SO₄ | 0.9 | 99 | 19 | 77 | 19 | n.a. | batch, extractant = toluene |
| 3.5 | 18.3 | 75 | H₂SO₄ | 0.9 | 100 | 8 | 87 | 8 | n.a. | batch, extractant = toluene |
| 4.1 | 24 | 75 | A15 | 0.002 | 95 | 46 | 43 | 48 | 0.7 | Batch, A15 |
| 4.2 | 24 | 75 | ASA | 0.36 | 56 | 35 | 15 | 64 | 1.7 | Batch, ASA |
| 5.1 | 24 | 75 | Re2O7 | 0.013 | 99 | 38 | 56 | 38 | 0.5 | Batch, Re2O7 |

### Example 6

C9-C20-Carbon-carbon coupled oligomers were produced by a process as described for above example 1.2. (acid catalysed oligomerisation of an aqueous solution comprising 35 wt % furfuryl alcohol, giving −60% C₉-C₂₀ carbon-carbon coupled oligomers). Most unreacted furfuryl alcohol was removed by washing the oligomers 5 times with water. Hereafter the C9-C20 carbon-carbon coupled oligomers were hydrogenated with a Ni/Al₂O₂ catalyst at 100° C. The product of this hydrogenation was distilled and the diesel fraction (boiling in the range from 170 to 370° C.) was isolated. This diesel fraction of the C9-C20 carbon-carbon coupled oligomers was subsequently blended into a base fuel at 10 vol %. The fuel blend containing 10 vol. % of C9-C20 carbon-carbon coupled oligomers was tested against key parameters in the European diesel specification EN 590. The results are summarized below in table 2. As illustrated in these results, the C9-C20 carbon-carbon coupled oligomers can be used to blend with a base fuel.

TABLE 2
Fuel blend containing 10 vol. % of C9-C20 carbon-carbon
coupled oligomers

| | | | Base fuel |
|---|---|---|---|
| | | | comprising |
| | | | 10 vol % |
| Property | EN 590 spec | Base Fuel | oligomers |
| Density at 15° C., | 820-845 | 835.5 | 852.5 |
| kg/m³- IP 365 | | | |
| Viscosity at 40° C., | 2.0-4.5 | 2.919 | 2.999 |
| mm²/s - IP 71 | | | |
| Cetane Number (IQT) - | 51 min | 65.2 | 61.0 |
| IP 498 | | | |
| Distillation - IP | 360 max | 183.7 | 160.0 |
| 123 IBP | | | |
| (° C.) | | | |
| 10% evap (° C.) | | 216.5 | 205.3 |
| 20% evap (° C.) | | 230.5 | 224.0 |
| 30% evap (° C.) | | 244.9 | 239.1 |
| 40% evap (° C.) | | 258.9 | 253.0 |
| 50% evap (° C.) | | 272.1 | 265.0 |
| 60% evap (° C.) | | 285.3 | 277.6 |
| 70% evap (° C.) | | 299.2 | 291.6 |
| 80% evap (° C.) | | 315.2 | 308.9 |
| 90% evap (° C.) | | 337.8 | 332.3 |
| 95% evap (° C.) | | 357.5 | 352.1 |
| FBP (° C.) | | 367.4 | 362.5 |
| Recovery, % vol | | 98.2 | 98.1 |
| Residue, % vol | | 1.3 | 1.3 |
| Loss, % vol | | 0.5 | 0.6 |
| Flash Point, ° C. - IP | 55 min | 69.0 | 64.5 |
| 170 | | | |
| Lubricity, microns - | 460 max | 359/421 | 328/390 |
| ISO 12156 | | | |
| Acidity, mgKOH/g - | | <0.05 | 0.06 |
| IP 139 | | | |
| Cloud Point, ° C. - IP | Climate & | −8 | −8 |
| 219 | season | | |
| CFPP, ° C. - IP 309 | dependant | −29 | −25 |

We claim:

1. A process for preparing a hydrocarbon or mixture of hydrocarbons comprising the steps of:
(i) hydrogenating furfural, 5-hydroxymethylfurfural or a mixture of furfural and 5-hydroxymethylfurfural thereby producing furfuryl alcohol, 2,5-furandimethanol or a mixture of furfuryl alcohol and 2,5-furandimethanol;
(ii) oligomerizing the alcohol or mixture of alcohols of step (i) in the presence of an acidic catalyst to provide a carbon-carbon coupled oligomer; and
(iii) hydrogenating the oligomer of step (ii).

2. The process of claim 1 wherein the oligomer product of step (ii) is extracted from the aqueous phase using an extraction solvent.

3. The process of claim 1 wherein 5-hydroxymethylfurfural is added to the alcohol or mixture of alcohols in the oligomerization step (ii).

4. The process of claim wherein step (ii) provides a product mixture containing unreacted furfuryl alcohol and/or unreacted 2,5-furandimethanol; C9-C20 carbon-carbon coupled oligomers; and C20+ carbon-carbon coupled oligomers and wherein one or more of unreacted furfuryl alcohol and/or unreacted 2,5-furandimethanol; C9-C20 carbon-carbon coupled oligomers; and/or C20+ carbon-carbon coupled oligomers from any product mixture are separated using one or more membranes.

5. The process of claim 1 wherein step (ii) comprises oligomerizing the alcohol or mixture of alcohols of step (i) in the presence of an acidic catalyst to provide a carbon-carbon coupled oligomer of formula (II) or (III) 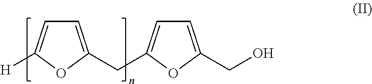
wherein n is an integer from 1 to 3, 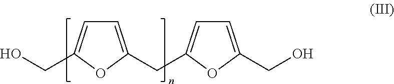
wherein n is an integer from 1 to 3;
and reacting the carbon-carbon coupled oligomer of formula (II) or (III) to convert one or more —CH₂OH group(s) into one or more ether and/or ester group(s).

6. The process of claim 1 wherein step (iii) comprises hydrogenating the oligomer of step (ii) to provide a product of formula (IV) 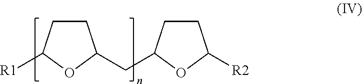
wherein n is an integer from 1 to 3; wherein R1 is H, —CH₂OH or —CH₃; and wherein R2 is H, —CH₂OH or —CH₃ and wherein at least one of R1 and R2 is —CH₂OH; and reacting the product of formula (IV) to convert one or more —CH₂OH group(s) into one or more ether and/or ester group(s).

7. A process for oligomerizing furfuryl alcohol, 2,5-furandimethanol or a mixture of furfuryl alcohol and 2,5-furandimethanol to prepare one or more carbon-carbon coupled oligomers comprising from 2 to 4 furan rings, comprising: contacting the alcohol or mixture of alcohols with an acidic catalyst wherein the alcohol conversion is in the range of from 20 to 95%.

8. The process of claim 7 wherein the carbon-carbon coupled oligomers comprise a compound of formula (I) or a compound of formula (II) or a compound of formula (III) 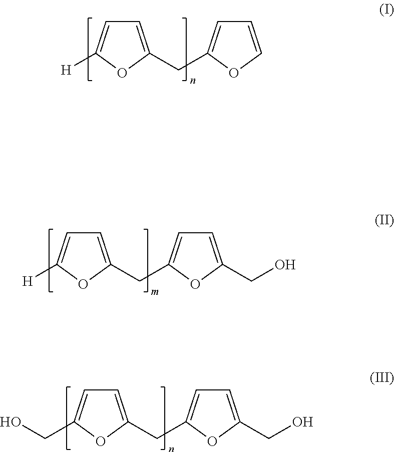
wherein n and m are each an integer from 1 to 3, or a mixture thereof.

9. The process of claim 7 wherein 5-hydroxymethylfurfural is fed to the furfuryl alcohol or mixture of alcohols in the oligomerization process.

10. A process for preparing a C₉-C₂₀ hydrocarbon comprising hydrogenating an oligomer of furfuryl alcohol, 2,5-furandimethanol or a mixture of furfuryl alcohol and 2,5-furandimethanol.

11. A process for preparing a fuel blending component comprising the process of claims 1.

12. A saturated ring product of formula (IV) 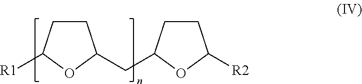
wherein n is an integer from 1 to 3; wherein R1 is H, —CH₂OH or —CH₃; and wherein R2 is H, —CH₂OH or —CH₃, with the proviso that at least one of R1 and R2 is —CH₂OH or —CH₃.

14. A saturated ring product of formula (VIII) 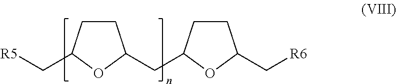
wherein n is an integer from 1 to 3, suitably 1, 2 or 3, and most preferably 2 or 3; wherein R5 is hydrogen, an —O—R group or an —O—C(O)—R group, wherein R is an alkyl group; and wherein R6 is hydrogen, an —O—R group or an —O—C(O)—R group, wherein R is an alkyl group; with the proviso that at least one of R5 and R6 is an —O—R group or an —O—C(O)—R group.

15. Use of a compound of formula (IV) 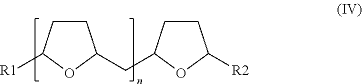
wherein n is an integer from 1 to 3; wherein R1 is H, —CH₂OH or —CH₃; and wherein R2 is H, —CH₂OH or —CH₃; or
a compound of formula (VIII) 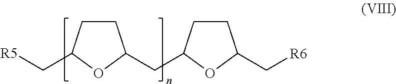
wherein n is an integer from 1 to 3; wherein R5 is hydrogen, an —O—R group or an —O—C(O)—R group, wherein R is an alkyl group; and wherein R6 is hydrogen, an —O—R group or an —O—C(O)—R group, wherein R is an alkyl group; with the proviso that at least one of R5 and R6 is an —O—R group or an —O—C(O)—R group, as a fuel component.
